(19) United States
(12) Patent Application Publication
(10) Pub. No.: US 2024/0096486 A1
(43) Pub. Date: Mar. 21, 2024

(54) SYSTEM AND METHOD FOR MONITORING WOUND THERAPY

(71) Applicant: FEELTECT LIMITED, Moycullen, Co. Galway (IE)

(72) Inventors: Andrew Cameron, Moycullen, Co. Galway (IE); Darren Burke, Moycullen, Co. Galway (IE)

(21) Appl. No.: 18/256,634

(22) PCT Filed: Dec. 15, 2021
(86) PCT No.: PCT/EP2021/086036

§ 371 Date: Jun. 8, 2023

(30) Foreign Application Priority Data
Dec. 15, 2020 (EP) 20214181.8

(51) Int. Cl. G16H 40/67; A61F 13/00; G16H 10/60; G16H 15/00; G16H 30/40

(52) U.S. Cl. CPC G16H 40/67; A61F 13/00; G16H 10/60; G16H 15/00; G16H 30/40; A61F 2013/00944; A61F 2013/00953; A61F 2013/0097

(57) ABSTRACT

The present invention relates to a system and method for monitoring, capturing, storing, processing, and enabling remote access of a plurality of data inputs pertaining to wound therapy of one or more subjects. The system as per the present invention comprises a wearable dedicated monitoring device, one or more user devices, a digital platform, a subject consent management means. The monitoring device is configured to receive a plurality of data inputs captured from one or more subjects undergoing wound therapy. The plurality of data inputs comprises one or more functional parameters of wound therapy, one or more modalities for wound therapy, and one or more factors impacting wound therapy. A unique identifier code is assigned by the monitoring device to specifically link the plurality of data inputs to each subject. Each of the plurality of data inputs is time stamped and based on the specific consent of the subjects, the data inputs are communicated to the user devices and the digital platform. The digital platform is configured to store, process, correlate, and enable remote access to the data inputs.

### FIELD

The present disclosure relates to a system and method for monitoring wound therapy, and more particularly to a system and method for monitoring, anonymizing, capturing, storing, processing a plurality of data inputs pertaining to wound therapy of one or more subjects.

### BACKGROUND

The treatment of wounds, for example, venous leg ulcers, can comprise numerous individual therapies, such as debridement, application of a primary/secondary dressing, application of a compression bandage, and possibly other advanced wound care therapies, such as application of biological agents or Negative Pressure Wound Therapy (NPWT). Each of the therapies have general functionalities, for example, dressings provide a barrier or absorb moisture/exudate, and compression bandages apply an interface pressure. If any of these functionalities are lost or reduced or compromised, there may be a need for informing the healthcare provider or the patient in self-management scenarios, so that necessary action can be taken to remedy the issues in said functionalities, in order to sustain effective wound care.

The need for a single system for monitoring wound therapy functionalities, relates to the potential for significant interplay within these functionalities, for example, the effect of compression pressure on dressing moisture caused by exudate, and their combined effect on wound healing. If these interplays are better understood, optimal combinations of therapies could be administered for more efficient healing with lesser resource requirements.

Another significant challenge in optimising wound therapy is evaluating the effect that different products have on the functionalities of each therapy. For example, within each of these therapies, there are a myriad of products that can be used with different variations of each product, for instance, different styles of compression bandage or different branded compression bandages of a particular style could be used. The choice of optimal products or combinations of products is hampered by a lack of an effective system for monitoring and correlating functionalities with the products that are used. Furthermore, often wounds are treated over multiple care settings by different care providers who may each use different products for each therapy. This may make it difficult to track the products to know what is effective for an individual patient, or to optimise product selection of an entire cohort of patients. Not only is this an issue for healthcare systems, but this is also potentially an issue for product manufacturers seeking to gain reliable data to enable product improvement and innovation.

Beyond products, there are various other factors that influence the functionalities of wound therapies, such as the characteristics of patients, for example, the wound type, patient size (for example BMI), wound size, compliance, tolerance to pain etc., environmental conditions, for example, temperature etc., or care settings, for example, provider type, facility type etc. It is important to monitor and record these factors along with the monitoring of functionalities of therapies and the modalities of therapies to determine if there are areas of improvement that can be made for individual patients, patient cohorts, or product development. Monitoring potential affecting factors of treatment is also important to understand if monitored functionalities for specific modalities of treatments are confined to sub-groups of patients or can be more generally attributed, therefore helping in product selection and patient stratification.

A challenge with monitoring wound therapy functionalities and their affecting factors is the chronic nature of wounds and the multiple care settings that they are treated in. For example, some settings may use different products, some may have different provider skillsets, some may have access to smart devices, some may have no access to internet. For these reasons, it is difficult to capture and input data about the different functionalities/product types/affecting factors of wound care treatment at each of the points on the patient pathway. Having a single dedicated point of capture for each of these functionalities/product types/affecting factors is important for continuity and accountability, particularly one that is impervious to variations in treatment modalities or different settings and agnostic to variations in affecting factors that may occur on the patient pathway (for example, capable or being used with multiple bandage types or dressing types).

A further challenge involved in capturing of health-related data is compliance to personal data privacy laws. While individual healthcare systems may have secure patient records, it is difficult to easily share these records between different systems. Not only is it difficult to share patient records between different healthcare systems, it is equally difficult to share data with external organisations, such as product manufacturers, which may also have a financial interest on the data or researchers, who may have an academic interest on the data. In addition, such data may be used by product manufacturers for improved safety and risk mitigation via field surveillance of products. One of the ways to avoid the breach of any sensitive or confidential data is to anonymise the collected data at the point of capture. However, in anonymising data in a collective system, it is still a requirement to have the opportunity to link the data to individual patients, or to patient episodes of treatment, in a systematic and consistent fashion, whilst not disclosing identifiable information within the system, so that treatments can be optimised on a patient level as well as on a cohort level. The need for both anonymisation and individualisation of data at the point of capture necessitates a dedicated collection point for collation, rather than using existing data input channels that already carry patient identifiable information or may be ambiguous as to which individual the data is related. Given the different forms of therapies that may be provided in the course of wound treatment, as well as the different settings in which therapies are provided, it can be a challenge to obtain consent for all different forms of collected data relating to wound treatment along the patient pathway. Similar to the challenge of monitoring wound therapy functionalities and their affecting factors in general, a way of addressing the challenge of obtaining consent is to have a single dedicated point of capture for all functionalities/product types/affecting factors whereby authorised consent can be provided for transmission (local or remote), external storage, or use of all data from that one point.

There are several inventions known in the art which monitor the functionality of wound therapies, such as using pressure sensors that monitor compression therapy or moisture sensors as disclosed in WO2018115461A1, and that which measures modalities for wound therapy as disclosed in WO2020127610A1. A few of these known inventions measure affecting factors in addition to wound therapy functionalities, such as by using pressure sensing devices that also account for temperature, as disclosed in US20150297437A1 or curvature of a patient's leg as disclosed in WO2018209100A1. There are also prior art inventions that describe the use of unique identification mechanisms or encryption to anonymise data such as the one disclosed in WO2018209100A1 and CN109069712A, as well as correlating different variables such as pressure or temperature as described in WO2018209100A1, or sensor values as described in CN109069712A.

Another patent publication US20060116904A1 describes the collection of wound treatment data using a plurality of input points. Other patent publications such as US2016/317077; US2020/352501 and US2019/262180 describe similar systems for wound treatment and collection of data, however these publications fail to provide the anonymization and individualization of data at the point of capture, leaving such systems susceptible to breach of data privacy or ambiguity in the source of data. Having multiple, variable, and distributed points of data input into the system also provides technical challenges with verification of data formats for the different fields/topics of data collected and carries the risk of false or corrupted data inputs, whether accidental or malicious. Authentication of collected data from different sources requires security protected (i.e. password protected) user accounts, removing the associated benefits of data anonymisation within the system (e.g. usability by multiple stakeholders without compromise of data privacy).

However, none of the prior art publications describe a means to collectively capture data inputs related to the functionalities, modalities, and affecting factors of wound therapies, in an anonymous, individualised, authorised/consented, and correlatable manner. Inventions known in the art that monitor the functionality of individual therapies do not have the ability to assess the interplay between different functionalities. Further, prior art devices that monitor therapy functionalities or affecting factors, do not monitor and capture information related to the modalities used for wound therapy which implies that with such prior art devices, there are no means to account for the products being used on a patient, which significantly impedes the ability for data driven product optimisation. It may be possible to manually input details of products into a database retrospectively, but during the treatment of chronic wounds that are cared for over multiple settings whereby different settings may use different products, there is a significant risk of data inputs being missed or being incorrect. Linking data with identifiable data would mean that it is not anonymised without further deidentification processes, thereby restricting the use where patient data privacy is paramount.

Further, none of the prior art inventions disclose means to capture the consent of authorised users for transmission, external storage, processing, or for viewing data related to wound therapy. This means that even if data captured by the prior art inventions are anonymous or deidentified, it may still be necessary to restrict their use by different stakeholders in accordance with the applicable data privacy laws. This makes it difficult to share data freely, especially to external healthcare systems.

There is therefore an unresolved and unfulfilled need in the art for a system and method which captures data inputs related to the functionalities, modalities, and affecting factors of wound therapies, in an anonymous, authorised and correlatable manner, and that which enables such captured data inputs to be uniquely linked to an individual, without compromising the individual's privacy or data usage rights.

### SUMMARY

The present invention enables automated capture, transfer, and processing of data relevant to optimisation of wound treatment protocols and products, for the purposes of maintaining or improving the standard of care received by individuals or cohorts, whilst abiding by personal data privacy and data usage rights, as set out in the appended claims.

The invention provides a system and method for linking data with unique identification codes at the point of capture or using a dedicated capture device. The invention overcomes the problem of presenting a risk of loss of data (e.g. in the case that unlinked data fails to be attributed to a patient), loss of privacy (e.g. in the case that data is linked with a non-dedicated unique identification code that compromises data privacy), or data corruption (e.g. in the case that data is linked via a non-dedicated unique identification code that belongs to the wrong individual or invalid data is deliberately linked to a non-dedicated unique identification code and transferred to the system).

In a preferred embodiment of the present invention a system for monitoring wound therapy is provided. The system comprises a wearable dedicated monitoring device having a memory means, and operably interfaced to a digital platform, one or more user devices, a plurality of sensors and a timestamp counter. The system further comprises a subject consent management means operably interfaced to the dedicated monitoring device and the one or more user devices. The consent management means enables patients to provide their consent for transfer, storage, or viewing of data, which will be stored within the dedicated monitoring device and automatically dictate the data usage accordingly.

The dedicated monitoring device is configured to receive a plurality of data inputs captured from one or more subjects undergoing wound therapy. The plurality of data inputs comprises one or more functional parameters of wound therapy, one or more modalities for wound therapy, and one or more factors impacting wound therapy. The timestamp counter is configured to assign a timestamp indicating the time of capture of each of the plurality of data inputs.

In an embodiment of the present invention, the one or more functional parameters of wound therapy includes the measure of pressure applied by compression therapy, the measure of moisture content of dressings, the pressure achieved by offloading devices in diabetic foot ulcer treatment, and the viscosity of wound exudate. The functional parameters of wound therapy are captured in regular intervals by a plurality of sensors including pressure sensors which may be pneumatic, optical, or piezoelectric, and moisture sensors which may be impedance based. The one or more modalities for wound therapy include the product type and product model used for wound therapy, type of wound therapy, and details of manufacturers of products used for wound therapy.

The details of devices or products are captured in random intervals by for example, scanning the product packaging using means such as barcode scanners and Quick Response (QR) code scanners. The details of modalities used for wound therapy can also be manually inputted to the dedicated monitoring device, for example via selection from a library of selection options. The one or more factors impacting wound therapy includes wound type, wound size, metrics of pain tolerance, temperature, pH, relative humidity, type of care provider to the subject, experience of care provider to the subject, limb size of the subject, and geographical location or clinical setting of the subject. Other factors that can be considered comprises one or more of the following BMI, diet, smoking, mobility, behaviours. The factors impacting wound therapy are captured via a combination of automated and manual means. For example, environmental conditions at wound therapy site such as temperature, pH, and relative humidity, may be captured in regular intervals using sensors, and inputs such as care provider details, physical attributes of the subject etc., may be inputted manually to the monitoring device in random intervals.

The dedicated monitoring device is further configured to assign a unique identifier code to uniquely link the plurality of data inputs to each of the one or more subjects. The unique identifier code, which is assigned to data at the point of capture, is anonymous and is specific to each subject or in other words each unique identifier code is associated with or tied to the subject or subjects it is assigned to. In an embodiment of the present invention the unique identifier code is unique and inherent to the dedicated monitoring device, taking a known format recognized by the digital platform, in which case the monitoring device is solely used by a single subject. The dedicated monitoring device also provides the opportunity to have inbuilt authentication protocols/certificates/tokens that validate the source of data, in addition to the unique identification codes displayed to users, for example acting as a multi-factor authentication for data entry. Ann advantage of having dedicated points of data capture is the use of associated authentication tokens inherent to the dedicated capture points (e.g. devices), which in addition to the appropriately formatted data (including separate unique identification codes), can be used as a means of verification that the data is originating from a valid or trusted source.

The plurality of data inputs is communicated to the one or more user devices and the digital platform subject to the consent of the subjects whose data inputs are communicated. The digital platform is configured to store, process, and correlate the communicated plurality of data inputs, and to also enable remote alerting or viewing of the plurality of data inputs. The stored data can be encrypted. The consent of the subjects is recorded through the subject consent management means which is adapted to enable specific consent of the subjects for each of the activities of communicating, storing, processing, and enabling remote access to, the plurality of data inputs. The consent is recorded in the monitoring device. The consent can be provided for the specific type and access, depending on the application required. For example, consent for functionalities or modalities may not require consent, as these could be seen as totally anonymous, whereas a hospital/patient may see things such as limb size, clinical setting, BMI, etc, which are related to affecting factors, as needing consent. In such a scenario it is envisaged to enable consent for the type of data, whereby data accessible and non-accessible data topics are segregated in the digital platform. Similarly, it may be that functionality and modalities may be shared with anyone, but a patient only wants to share affecting factors with their healthcare provider. In such a scenario it is necessary to consent for the type of data and the data access on a user-specific basis. In one embodiment where consent can be provided for searching information in the database related to a unique device identification, in this scenario, the data type is the device identification, the data use is search, and the data access is anyone in possession of the unique identification number.

In one embodiment the consent means is related to data type, data use, and data access. Type being related to functionalities, modalities, affecting factors, and unique identification number. Use being related to transfer, searching, processing, storage, display, and downstream purposes. Access being related to the nature of the user that is able to use a given data type, or specifying the relationship of the user to the accessible data.

The plurality of data inputs communicated to the digital platform comprises the chronologically ordered collective data of the functionality of wound therapies, the modalities of wound therapies, and the factors that impact functionalities.

The plurality of data inputs is linked by unique identifier codes that are anonymous, and specific and unique for each dedicated monitoring device, which in turn is unique to each subject, where the dedicated monitoring device is only applied to a single subject. The unique identifier codes hence allow communication, storage, and processing of data inputs without disclosing personal information of subjects.

In an embodiment of the present invention, the digital platform comprises a cloud database platform and one or more user interfaces operably interfaced to the cloud database platform. It will be appreciated that individual sensing units interfacing with the dedicated monitoring device can be configured to have their own unique identification codes, for tracking purposes or identifying when a sensor is changed for a particular dedicated monitoring device/patient. However, the unique identification code for the dedicated monitoring device will remain the primary key for the main data relationship table within the cloud database.

The digital platform is configured to assess and process the plurality of data inputs data to elucidate the interplay between each of the plurality of data inputs. The plurality of data inputs communicated to the digital platform can be correlated with data captured within datasets on wound healing outcomes, or patient health records, for example to develop predictive algorithms, to further optimise treatment provision for individual subjects or subject cohorts.

In an embodiment of the present invention, the digital platform is operably interfaced to an electronic health record system. This enables healthcare personnel to gain insights from the data inputs processed by the digital platform which in turn enables improvisation and optimization of wound therapy protocols.

In an embodiment the digital platform is operably interfaced to an EHR system, one or more user devices, or external digital platforms monitoring the wound condition. Such a configuration is advantageous where a “treatment” digital platform is combined with the digital platform of a wound “healing” monitoring system.

In a preferred embodiment of the present invention, a method for wound therapy is provided. The method is implemented by a wearable dedicated monitoring device operably interfaced to a digital platform and one or more user devices. The method comprises the first step of capturing a plurality of data inputs from at least one subject. Said plurality of data inputs comprises one or more functional parameters of wound therapy, one or more modalities for wound therapy, and one or more factors impacting wound therapy. A timestamp is assigned to each of the plurality of data inputs to indicate the time of capture of each of the plurality of data inputs. The timestamp can be applied on the dedicated device side or on the digital platform. Further, a unique identifier code is assigned to the plurality of data inputs, at the point of capture, collected from each subject, which enables linking of the plurality of data inputs specifically to each subject without divulging personal information of the subject. The captured plurality of data inputs is then communicated to the one or more user devices and to the digital platform based on the consent of the one or more subjects for such communication. The consent of the subject is recorded for each of the acts of communicating, storing, processing, correlating, and enabling remote access to the plurality of data inputs. Based on the consent of each of the subjects, the plurality of data inputs is stored, processed, and correlated by the digital platform. The digital platform is further configured to allow remote access to the plurality of data inputs based on the consent of the subject.

The ability to capture, store, and transmit the combined data relating to the functionalities of different forms of wound treatments provides opportunities to rapidly identify and address any deficiencies in any of such treatments, which is also an advantage over prior art inventions that individually monitor wound therapy functionalities and may miss deficiencies elsewhere. Having data relating to the wound treatment received by the subject stored on the dedicated monitoring device provides opportunities for overcoming challenges of continuity of care between different healthcare settings by acting as a wound treatment “passport” that remains with the subject. It also provides significant opportunities for correlating the modality of the treatment with treatment functionalities which helps healthcare service providers to take informed buying decisions and enables product manufacturers to improve and optimize their products.

By capturing data on the functionality of wound therapies, the modalities of wound therapies, and the factors that impact the functionalities of the therapies, and subsequently applying a unique identifier code to link that captured data, the present invention enables a subject-level assessment, on an individual basis or as part of a cohort, of interplay between different data, which can be used by multiple stakeholders to optimise the therapy provided to a single subject or cohort of subjects. Such optimisation may result in reduced healing times, improved efficiencies of care (e.g. fewer patient visits), reduced costs of treatment, and greater abilities to allocate care resources where required. This ability to optimise therapy becomes more powerful when the data within the system is combined with the data on healing outcomes, such as the data collected by healthcare systems for a particular patient or cohort of subjects, to evaluate how wound therapies affect wound healing. For example, this may be achieved by the subject providing the healthcare system with their own device's unique identification code, which the healthcare system can then use to retrieve consented data from the digital platform and correlate internally with their electronic health record system. Data from the digital platform may be shared with external systems via an application programming interface (API). Conversely, deidentified patient information could also be extracted from the external healthcare systems and incorporated into the digital platform by manually inputting data into the dedicated monitoring device or by using an API to match select fields of interest, with the appropriate consent provided.

The present invention may also be used to make informed decisions about the type of products that may be provided to subjects to achieve desired functionalities, individually or in combination with other products, taking into context the factors that may impact said functionalities. The combined information on treatment functionalities will also better inform whether a subject's treatment pathway needs intervention, for example, if there are issues with either compression pressure or dressing moisture, the care provider can be notified and they can make a decision on course of action. This is an improvement on devices that measure these functionalities separately or that do not capture extenuating factors.

The present invention can also be a unique data collection point for an individual subject receiving wound care therapies, which can be used independently and without the need for continuous connectivity to a wider network. While the digital platform enables monitoring of entire subject cohorts, the device described in the current invention has the storage capacity to hold all the data on therapy functionality, modalities, and affecting factors for an individual subject. This means that the data can be retrieved for an individual subject via network communication means such as Bluetooth, to a smart device hosting the user interface of the digital platform, irrespective of the internet connectivity, which is important in community settings.

In one embodiment data can be streamed directly from the dedicated monitoring device to the digital platform using NB IoT, reducing the on-device storage capacity requirements whilst not relying on external communication networks within the different care settings.

There is also provided a computer program comprising program instructions for causing a computer program to carry out the above method which may be embodied on a record medium, carrier signal or read-only memory.

The present invention hence provides a robust solution to problems identified in the art. Other advantages and additional novel features of the present invention will become apparent from the subsequent detailed description.

### BRIEF DESCRIPTION OF DRAWINGS

The invention will be more clearly understood from the following description of an embodiment thereof, given by way of example only, with reference to the accompanying drawings, in which:

FIG. 1 is a schematic diagram illustrating a system as per a preferred embodiment of the present invention.

FIG. 2 is a schematic diagram illustrating the subject consent management means as per a preferred embodiment of the present invention.

FIG. 3 is a schematic diagram illustrating a system as per a preferred embodiment of the present invention.

FIG. 4 is a schematic diagram illustrating a system as per a preferred embodiment of the present invention.

FIG. 5 is a schematic diagram illustrating a system as per a preferred embodiment of the present invention.

FIG. 6 is a schematic diagram illustrating a system as per a preferred embodiment of the present invention.

FIG. 7 is a flow diagram illustrating a method as per a preferred embodiment of the present invention.

### DETAILED DESCRIPTION OF DRAWINGS

The present invention relates to a system and method for monitoring wound therapy, and more particularly to a system and method for monitoring, capturing, storing, processing, and enabling remote access of a plurality of data inputs pertaining to wound therapy of one or more subjects.

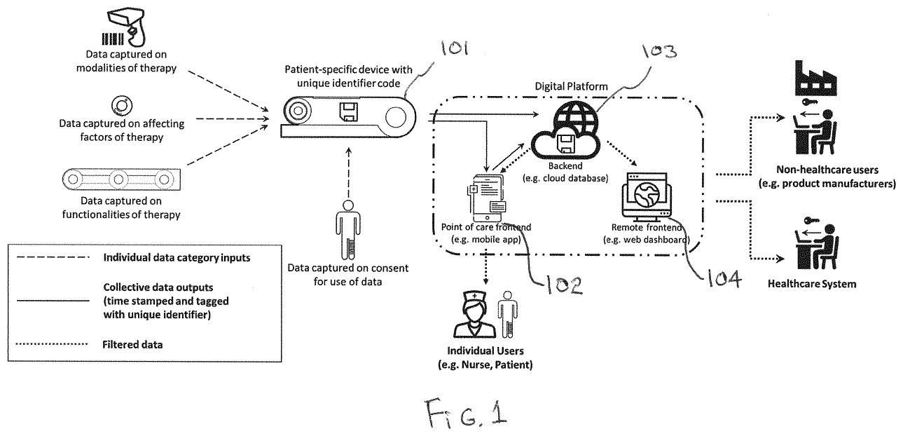

FIG. 1 illustrates a system according to a preferred embodiment of the present invention. The system comprises a wearable monitoring device 101 operably interfaced to a digital platform or database 103 and one or more user devices 102. The monitoring device is further operably interfaced with an optional time stamp counter and a plurality of sensors. The time stamp counter can be configured with the digital platform. The system further comprises a subject consent management means operably interfaced with the monitoring device 101 and the one or more user devices 102. The monitoring device 101 has a memory means which has a plurality of instructions stored thereon which in turn configures the monitoring device 101 to receive a plurality of data inputs from one or more subjects undergoing wound therapy. Said plurality of data inputs comprises one or more functional parameters of wound therapy, one or more modalities for wound therapy, and one or more factors impacting wound therapy. In an embodiment of the present invention, the wound therapy relates to treatment for disorders such as venous leg ulcers, lymphodema, DVT, diabetic foot ulcers, post venous ablation, and post varicose veins treatment.

The plurality of data inputs is captured either by the plurality of sensors or are manually inputted to the monitoring device 101.

The plurality of sensors comprises one or more pressure sensors, one or more moisture sensors, one or more barcode scanners, one or more quick response code scanners, one or more temperature sensors, one or more pH sensors, one or more optical scanners, one or more relative humidity sensors, and one or more position sensors. Other sensors can be provided such as motion sensors (accelerometers, vibration, gyroscopic), biomolecule sensors, gas sensors, and acoustic sensors. In an embodiment of the present invention, the monitoring device 101 communicates with the plurality of sensors through electrodes, or wirelessly for example using RFID of NFC. The monitoring device 101 is adapted to receive sensor signals, process the received signals, for example, via a calibration, and store the received signals. The monitoring device is also capable of wirelessly transmitting and/or receiving processed signals, via protocols such as Bluetooth, WiFi, BLE, Zigbee, Z-Wave, 6LoWPAN, NFC, WiFi Direct, GSM, LTE, LoRa, NB-loT, and LTE-M. Transmitted signals may include live readings from sensors or data stored in the monitoring device 101.

The one or more functional parameters of wound therapy includes the measure of pressure applied by compression therapy, the position of measurement, the measure of moisture content of dressings, and the viscosity of wound exudate. The functional parameters are captured in regular intervals using the plurality of sensors. It will be appreciated that the list is not exhaustive with other potential functionalities of therapy, for example suction of negative pressure wound therapy, pressure of unloading diabetic foot inserts and so on.

The one or more modalities for wound therapy can be interpreted broadly and include the product type and product model used for wound therapy, topical agents, type of wound therapy, and details of manufacturers of products used for wound therapy. These data inputs are captured in random intervals generally using product scanners such as quick response code scanners, and barcode scanners. The data inputs can be provided on a dropdown menu on an interface of the digital platform, transmitted to monitoring device via user device. The captured product specific data inputs are cross-referenced by the monitoring device 101 with a library/registry of product identification information.

The one or more factors impacting wound therapy includes wound type, wound size, a metric of pain tolerance, temperature, pH, relative humidity, type of care provider to the subject, experience of care provider to the subject, limb size of the subject, and geographical location of the subject. Data inputs such as temperature, pH, location of the subject, pain levels, and relative humidity are captured using the plurality of sensors such as the temperature sensors, pH sensors, position sensors, optical scanners, and relative humidity sensors. Data inputs such as type and experience of care provider, limb size of the subject and other patient details is inputted manually in random intervals.

The dedicated monitoring device 101 assigns a unique identifier code, which holds a format known to the digital platform, to the plurality of data inputs captured from each subject. The unique identifier code specifically links the plurality of data inputs to each subject, or in other words, the unique identifier code assigned for one subject is different from that assigned to another subject. In an embodiment of the present invention the unique identifier code is unique and inherent to the dedicated monitoring device 101 and the dedicated monitoring device 101 is solely used by a single subject. In said embodiment, the unique identifier code is specific to the monitoring device 101, and all the data inputs captured by the dedicated monitoring device 101 are labelled with the unique identifier code. The format of the unique identification codes is consistent and therefore recognisable within the digital platform. The captured data inputs can also be timestamped by a timestamp counter to chronologically manage the data for any unique device datapoint.

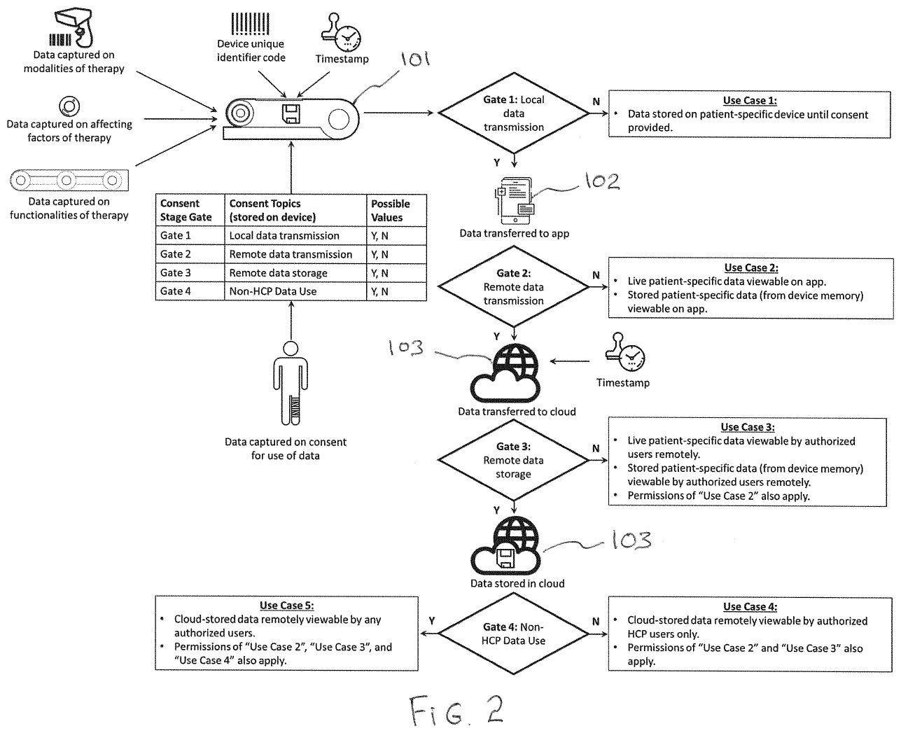

The monitoring device 101 is further configured to communicate the captured plurality of data inputs to the one or more user devices 102 and the digital platform 103, based on the consent of the one or more subjects recorded through the subject consent management means or module. The subject consent management means is operably interfaced with the one or more user devices 102 and the monitoring device 101. In an embodiment of the present invention, the consent of the subject is recorded through the user device 102 and is further communicated to the monitoring device. FIG. 2 illustrates a consent management means as per a preferred embodiment of the present invention. As shown, the consent of the subject is recorded for each of the stages of communication, storage, processing, and external access for the plurality of data inputs. The consent management allows for a gated system which enables each subject to selectively permit the different ways in which their data can be used, stored, processed, or accessed. The consent provided by the one or subjects are recorded and maintained within the monitoring device 101. For example, this may be achieved using an MQTT protocol with a series of topics related to the different forms of data, including the unique identification code of the dedicated monitoring device, which is the primary key for collated data within the digital platform database.

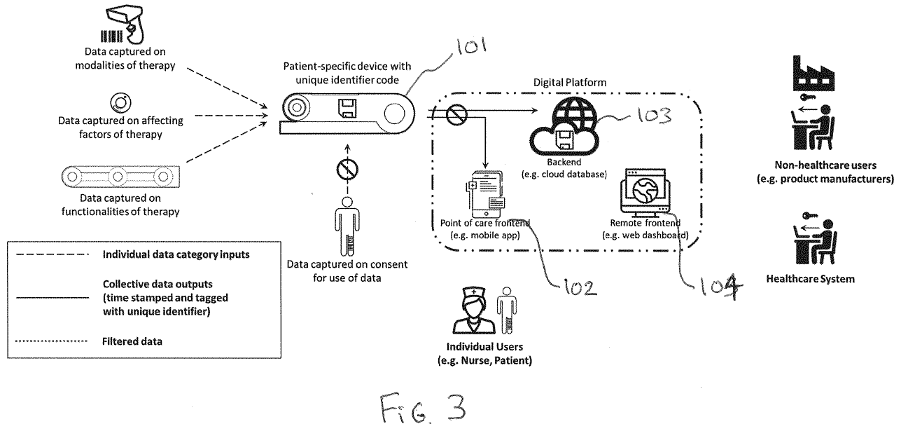

Referring to FIG. 2 and FIG. 3, consent for gate 1 relates to communication of the plurality of data inputs to the one or more user devices 102 from the monitoring device 101. Consent for gate 1 allows the plurality of data inputs to be communicated to the user device and displayed to the subject and to the care givers of the subject through the user device 102. If consent for Gate 1 is not provided, the data remains in or on the monitoring device 101 as illustrated in FIG. 3.

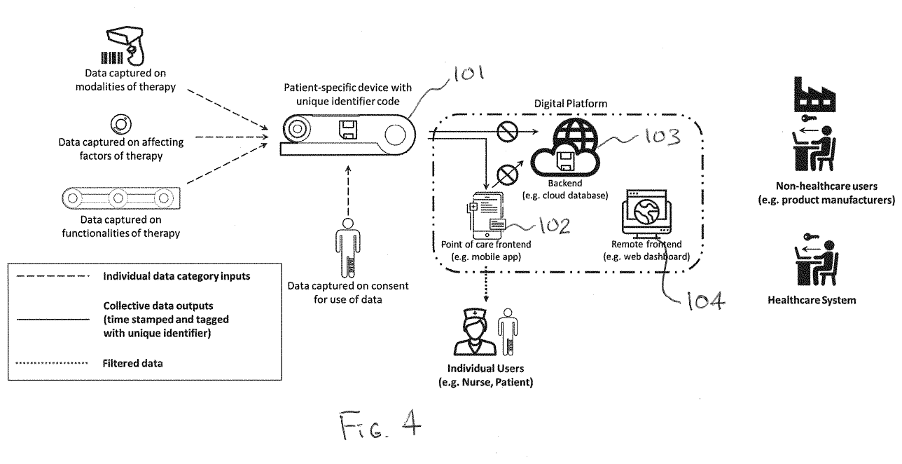

Referring to FIG. 2 and FIG. 4, consent by the one or more subjects at gate 2 enables communication of the plurality of data inputs from the monitoring device 101 to the digital platform 103. If consent at gate 2 is not provided, the plurality of data inputs will be communicated only to the user devices and will only be viewable therein. In an embodiment of the present invention, based on consent at gate 2, the user devices 102 are adapted to communicate the plurality of data inputs to the digital platform 103. The digital platform 103 comprises a cloud database platform, and one or more user interfaces 104 operably interfaced to the cloud database platform. The digital platform is configured to enable remote viewing of the plurality of data inputs through the one or more user interfaces 104.

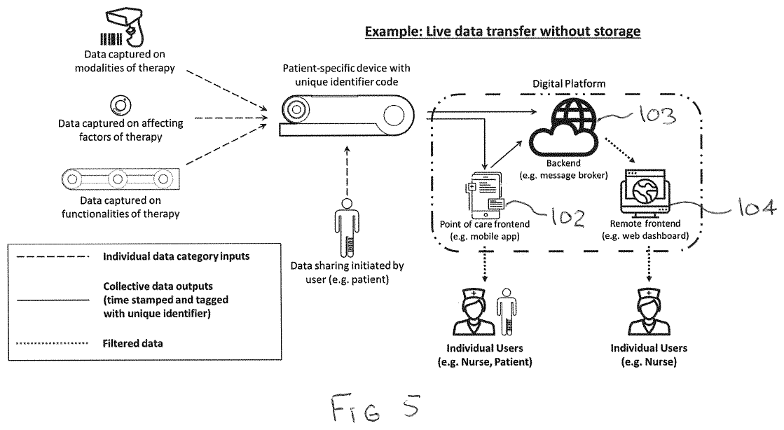

Referring to FIG. 2 and FIG. 5, consent by the one or more subjects at gate 3 enables the plurality of data inputs to be stored in the digital platform 103 for later access and use for any suitable data processing purposes. Without consent of the subject at gate 3, the plurality of data inputs is not stored in the digital platform 103 and are only remotely viewable as live data by authorized personnel through the digital platform 103 during the period of transmission and is no longer viewable once the transmission ends.

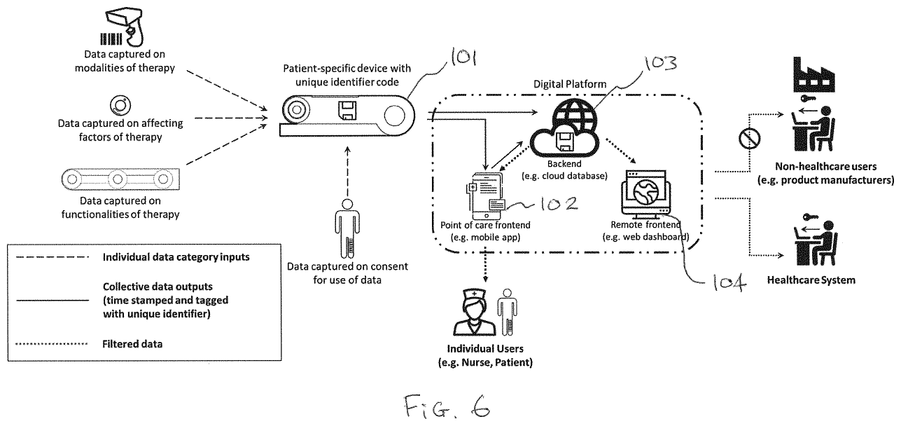

Referring to FIG. 2 and FIG. 6, consent by the one or more subjects at gate 4 enables remote access to the plurality of data by health care professionals and non-health care professionals and entities such as medical device manufacturers and data processing companies. This enables the plurality of data inputs to be processed and used for purposes related to improvement of wound treatment.

The privacy of subjects is safeguarded by linking the plurality of data inputs of each subject to a unique identifier code as aforementioned. This enables the plurality of data inputs communicated, for example to the digital platform 103, to be searched or collated at an individual level using the unique identifier code. The consent management means is also adapted to enable a subject to allow authorized users, for example health care professionals, to remotely search for the specific data inputs related to said subject using the unique identifier code. The different fields of data, including the unique identification code, time stamps, consent, and monitored data, can all be assigned as topics using MQTT data transport protocols, for transmission and storage within the digital platform.

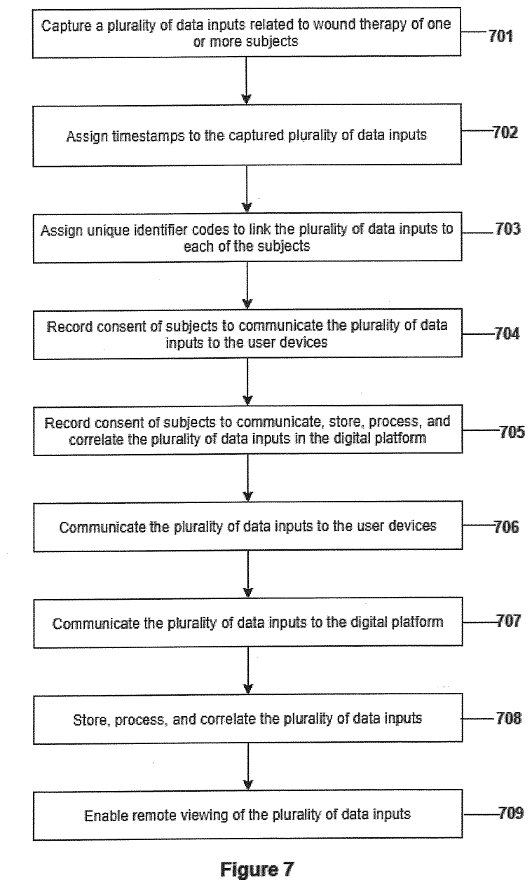

FIG. 7 illustrates a method as per a preferred embodiment of the present invention. The method is implemented by a wearable dedicated monitoring device operably interfaced to one or more user devices and a digital platform. The method comprises the first step of capturing a plurality of data inputs related to wound therapy from one or more subjects, whereby the dedicated monitoring device acts as a hub for data collation and linkage at the point of collection 701. In an embodiment of the present invention, the wound therapy relates to treatment of disorders such as venous leg ulcers, lymphodema, DVT, post venous ablation, diabetic foot ulcers and post varicose veins treatment. The plurality of data inputs comprises one or more functional parameters of wound therapy, one or more modalities for wound therapy, and one or more factors impacting wound therapy. The one or more functional parameters of wound therapy includes the measure of pressure applied by compression therapy, the measure of moisture content of dressings, the pressure achieved by offloading devices and the viscosity of wound exudate. The one or more modalities for wound therapy include the product type and product model used for wound therapy, type of wound therapy, and details of manufacturers of products used for wound therapy. The one or more factors impacting wound therapy includes wound type, wound size, a metric of pain tolerance, temperature, pH, relative humidity, type of care provider to the subject, experience of care provider to the subject, limb size of the subject, and geographical location of the subject

Timestamps are assigned to each of the plurality of data inputs, either at the dedicated monitoring device or within the digital platform, to indicate the chronological order in which the data inputs were captured 702. A unique identifier code is assigned to plurality of data inputs captured from each subject 703. This enables specific linking of the plurality of data inputs to each subject wherein each subject is associated with a unique identifier code. In doing so, data is both anonymized and linked with an individual at the point of data capture. In an embodiment of the present invention, the unique identifier code is unique to the monitoring device in which case the monitoring device is used solely by a single subject. Other unique identification codes may also relate to the sensor devices that interface with the dedicated monitoring device. Further, the consent of the one or more subjects is recorded to communicate the plurality of data inputs to the one or more user devices 704, and to communicate, store, process, and correlate the plurality of data inputs in the digital platform 705. The consent of each subject is recorded to enable each of the acts of communicating, storing, processing, and correlating the plurality of data inputs. Based on the consent of the subjects, the plurality of data inputs is communicated to the one or more user devices 706, and to the digital platform 707.

The digital platform is configured to store, process, correlate, and enable remote viewing of the plurality of data inputs subject to the consent of the subjects.

Although the present invention has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present invention as defined.

Further, a person ordinarily skilled in the art will appreciate that the various illustrative method steps described in connection with the embodiments disclosed herein may be implemented using electronic hardware, or a combination of hardware, firmware and software. To clearly illustrate this interchangeability of hardware and a combination of hardware, firmware and software, various illustrations and steps have been described above, generally in terms of their functionality. Whether such functionality is implemented as hardware or a combination of hardware and software depends upon the design choice of a person ordinarily skilled in the art. Such skilled artisans may implement the described functionality in varying ways for each particular application, but such obvious design choices should not be interpreted as causing a departure from the scope of the present invention.

The method described in the present disclosure may be implemented using various means. For example, the system described in the present disclosure may be implemented in hardware, firmware, software, or any combination thereof. For a hardware implementation, the processing units, or processors(s) or controller(s) may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

For a firmware and/or software implementation, software code may be stored in the memory means and executed by a processor. The memory means may be implemented within the processor unit or external to the processor unit. As used herein the term “memory” refers to any type of volatile memory or non-volatile memory.

The embodiments in the invention described with reference to the drawings comprise a computer apparatus and/or processes performed in a computer apparatus. However, the invention also extends to computer programs, particularly computer programs stored on or in a carrier adapted to bring the invention into practice. The program may be in the form of source code, object code, or a code intermediate source and object code, such as in partially compiled form or in any other form suitable for use in the implementation of the method according to the invention. The carrier may comprise a storage medium such as ROM, e.g. a memory stick or hard disk. The carrier may be an electrical or optical signal which may be transmitted via an electrical or an optical cable or by radio or other means.

In the specification the terms “comprise, comprises, comprised and comprising” or any variation thereof and the terms include, includes, included and including” or any variation thereof are considered to be totally interchangeable and they should all be afforded the widest possible interpretation and vice versa.

The invention is not limited to the embodiments hereinbefore described but may be varied in both construction and detail.

1. A system for monitoring wound therapy, the system comprising:
a wearable dedicated monitoring device having a storage means, the monitoring device operably interfaced to a digital platform, at least one device, and a plurality of sensors;
a subject consent management means operably interfaced to the monitoring device and the at least one device;
wherein the memory means has a plurality of instructions stored thereon which configures the dedicated monitoring device to:
receive a plurality of data inputs captured from at least one subject undergoing wound therapy, the plurality of data inputs comprising one or more functional parameters of wound therapy, one or more modalities for wound therapy, and one or more factors impacting wound therapy;
assign a unique identifier code to uniquely link the captured plurality of data inputs to the at least one subject at the point of capture; and
communicate the received plurality of data inputs to the at least one user device and to the digital platform, said communication based on the consent of each of the at least one subject, said consent recorded through the subject consent management means, wherein the digital platform is configured to store, process, and correlate the communicated plurality of data inputs; and the system comprises a timestamp counter is configured to assign a timestamp indicating the time of capture of each of the plurality of data inputs.

2. The system as claimed in claim 1, wherein the digital platform comprises a cloud database platform, and one or more user interfaces operably interfaced to the cloud database platform.

3. The system as claimed in claim 1 and claim 2 wherein the digital platform is configured to enable point-of-care or remote viewing of the plurality of data inputs through the one or more user interfaces.

4. The system as claimed in any preceding claim, wherein the digital platform is operably interfaced to an electronic health record system and to the one or more user devices.

5. The system as claimed in any preceding claim, wherein the one or more functional parameters of wound therapy includes the measure of pressure applied by compression therapy, the measure of moisture content of dressings, the pressure achieved by offloading devices, and the viscosity of wound exudate.

6. The system as claimed in any preceding claim, wherein the one or more modalities for wound therapy includes the product type and product model used for wound therapy, type of wound therapy, and details of manufacturers of products used for wound therapy.

7. The system as claimed in any preceding claim, wherein the one or more factors impacting wound therapy includes wound type, wound size, a metric of pain tolerance, temperature, pH, relative humidity, type of care provider to the subject, experience of care provider to the subject, limb size of the subject, and geographical location of the subject.

8. The system as claimed in any preceding claim, wherein at least one of the plurality of data inputs is captured by the plurality of sensors.

9. The system as claimed in any preceding claim, wherein at least one of the plurality of data inputs is manually inputted to the monitoring device.

10. The system as claimed in claim 8, wherein the plurality of sensors comprises one or more pressure sensors, one or more moisture sensors, one or more barcode scanners, one or more quick response code scanners, one or more temperature sensors, one or more pH sensors, one or more optical scanners, one or more relative humidity sensors, one or more position sensors, and motion sensors (accelerometers, vibration, gyroscopic), biomolecule sensors, gas sensors, and acoustic sensors.

11. The system as claimed in any preceding claim, wherein the unique identifier code is unique to the dedicated monitoring device, and wherein the monitoring device is solely used by a single subject.

12. The system as claimed in any preceding claim, wherein the consent management means is adapted to record specific consent of the one or more subjects for each of the acts of communicating, storing, processing, and enabling remote viewing of, the plurality of data inputs.

13. A system for monitoring wound therapy, the system comprising:
a wearable dedicated monitoring device having a memory means, the monitoring device operably interfaced to a plurality of sensors, the memory means has a plurality of instructions stored thereon which configures the monitoring device to:
receive a plurality of data inputs captured from one or more subjects undergoing wound therapy, the plurality of data inputs comprising one or more functional parameters of wound therapy, one or more modalities for wound therapy, and one or more factors impacting wound therapy; and
assign a unique identifier code to uniquely link the captured plurality of data inputs to each of the one or more subjects;
and the system comprises a timestamp counter is configured to assign a timestamp indicating the time of capture of each of the plurality of data inputs.

14. A method for monitoring wound therapy implemented by a wearable dedicated monitoring device operably interfaced to at least one user device and a digital platform, the method comprising the steps of:
a) capturing a plurality of data inputs from at least one subject undergoing wound therapy, the plurality of data inputs comprising one or more functional parameters of wound therapy, one or more modalities for wound therapy, and one or more factors impacting wound therapy;
b) assigning a timestamp indicating the time of capture of each of the plurality of data inputs;
c) assigning a unique identifier code to uniquely link the plurality of data inputs captured in step (a) to each of the one or more subjects;
d) recording consent of the one or more subjects to communicate the plurality of data inputs to the one or more user devices;
e) recording consent of the one or more subjects to communicate, store, process, and correlate, the plurality of data inputs in the digital platform;
f) communicating the plurality of data inputs captured in step (a) to the one or more user devices based on the consent recorded in step (d);
g) communicating the plurality of data inputs captured in step (a) to the digital platform based on the consent recorded in step (e); and
h) storing, processing, and correlating the plurality of data inputs communicated in step (g).
